(19) United States
(12) Patent Application Publication
(10) Pub. No.: US 2008/0255231 A1
(43) Pub. Date: Oct. 16, 2008

(54) POLYMORPHS OF RIVASTIGMINE HYDROGENTARTRATE

(76) Inventors: Arjanne Overeem, Beuningen (NL); Henar Tejedor Vinent, Lent (NL)

Correspondence Address: SYNTHON IP INC, 7130 HERITAGE VILLAGE PLAZA, STE 202, GAINESVILLE, VA, 20155, US

(21) Appl. No.: 12/044,626

(22) Filed: Mar. 7, 2008

Related U.S. Application Data
(60) Provisional application No. 60/894,071, filed on Mar. 9, 2007.

(51) Int. Cl. A61K 31/27; C07C 271/06; A61P 25/28

(52) U.S. Cl. 514/490; 560/115

(57) ABSTRACT

A crystalline rivastigmine hydrogentartrate of Form II and an amorphous rivastigmine hydrogentartrate of Form III.

This application claims the benefit of priority under 35 U.S.C. §119(e) from U.S. Provisional Application Ser. No. 60/894,071, filed Mar. 9, 2007, the entire contents of which are incorporated herein by reference.

### BACKGROUND OF THE INVENTION

1. Field of the Invention

The present invention relates to new forms of [3-[α-(Dimethylamino)-ethyl]phenyl]-N-methylethylcarbamate hydrogentartrate, a useful pharmaceutically-active agent, to compositions and pharmaceuticals containing the same, and to methods of making and using the same.

2. Description of the Prior Art

Rivastigmine is a generic name for the compound (S)-[3-[α-(Dimethylamino)-ethyl]phenyl]-N-methylethylcarbamate of the formula (I):

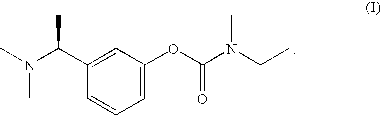

Rivastigmine (I) is a pharmaceutically active compound that acts as a reversible, brain-selective acetylcholinesterase inhibitor. In existing medicinal products, rivastigmine is marketed as a salt with L-tartaric acid, i.e., rivastigmine hydrogentartrate. Rivastigmine hydrogentartrate is indicated for the symptomatic treatment of mild to moderately severe Alzheimer's disease.

Rivastigmine hydrogentartrate is reported to be a white to off-white, fine crystalline powder that is very soluble in water, soluble in ethanol and acetonitrile, slightly soluble in n-octanol, and very slightly soluble in ethyl acetate. The reported melting point of rivastigmine hydrogentartrate is 123-125° C., specific rotation α[D/20]=+4.7 (c=5 in ethanol).

The compound of formula (I) has been generically disclosed, e.g., in EPB 193926 and U.S. Pat. No. 4,948,807. The resolution of the racemic equivalent of the compound of formula (I) into enantiomers and the conversion of the resolved (S)-enantiomer into a hydrogentartrate salt (rivastigmine hydrogentartrate) has been disclosed in GB 2203040 (U.S. Pat. No. 5,602,176) and in WO 03-101917. Processes of isolating the solid state rivastigmine hydrogentartrate were disclosed in WO 2004-037771, CN 1486973, WO 2005/061446, and WO 2006/048720.

Conventionally, it is unknown whether solid state rivastigmine hydrogentartrate may exist as different polymorphic modifications. The discovery of a new polymorph of a pharmaceutically active ingredient increases the number of options available to a pharmaceutical researcher for the formulation of a corresponding drug product. The discovery of a new polymorph of a pharmaceutically active ingredient therefore would be advantageous.

### SUMMARY OF THE INVENTION

An aspect of the present invention provides for a crystalline form of rivastigmine hydrogentartrate denoted as the Form II. Form II is characterized by characteristic x-ray powder diffraction (XRPD) peaks at angles of about 9.6, 11.4, 13.4, 19.2, 20.2, and 22.4° 2θ+/−0.1°, such as angles of about 9.6, 11.4, 13.4, 15.7, 19.2, 20.2, 22.4, 24.8, and 29.6° 2θ+/−0.1° and angles of about 9.6, 11.4, 13.4, 14.2, 15.7, 19.2, 20.2, 22.4, 24.8, 26.8, 29.6, 31.3, and 33.7° 2θ+/−0.1 °. Preferably, the XRPD pattern of the rivastigmine hydrogentartrate Form II does not include peaks at angles of about 5.2, 14.8, 18.8, 20.6, and 21.2° 2θ+/−0.1°. Form II is also characterized IR absorbance peaks at 3455, 3422, 3321, 1717, 1699, 1655, 1406, 1338, and 948 cm⁻¹+/−5 cm⁻¹. A substantially pure Form II exhibits an XRPD pattern substantially as shown in FIG. 3 and an IR absorbance spectrum substantially corresponding to FIG. 4.

Another aspect of the present invention provides an amorphous solid state form of rivastigmine hydrogentartrate denoted as Form III. A substantially pure Form III exhibits an XRPD pattern substantially as shown in FIG. 5.

A further aspect of the present invention provides processes of making the above Forms II and III of rivastigmine hydrogentartrate.

Additional aspects of the present invention provide compositions, including mixtures and pharmaceutical compositions, comprising the above Form II and/or Form III of rivastigmine hydrogentartrate, optionally in an admixture with the known Form I thereof, and uses thereof as a medicament.

### BRIEF DESCRIPTION OF THE DRAWINGS

FIG. 1 shows the XRPD pattern of conventional rivastigmine hydrogentartrate Form I;

FIG. 2 shows the IR absorbance spectrum for conventional rivastigmine hydrogentartrate Form I;

FIG. 3 shows the XRPD pattern of rivastigmine hydrogentartrate Form II;

FIG. 4 shows the IR absorbance spectrum for rivastigmine hydrogentartrate Form II; and

FIG. 5 shows the XRPD pattern of rivastigmine hydrogentartrate Form III.

### DETAILED DESCRIPTION OF THE INVENTION

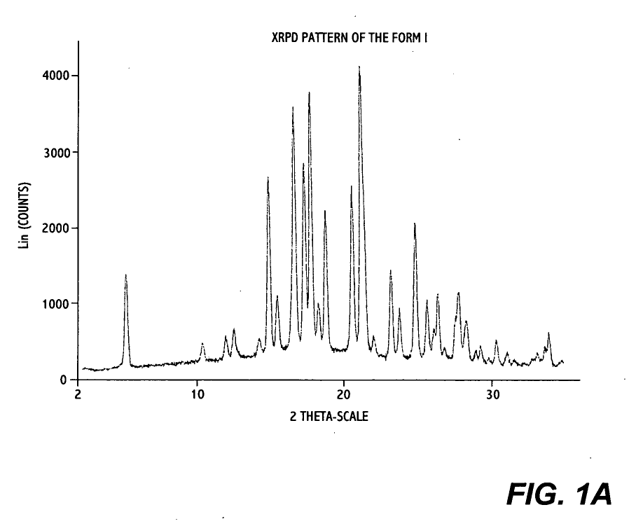

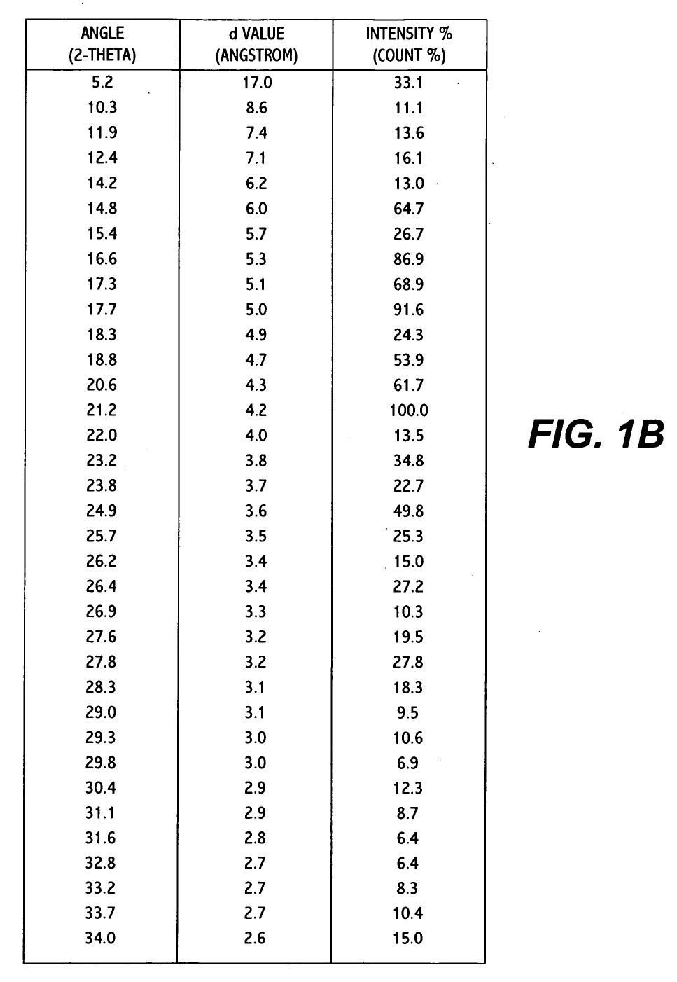

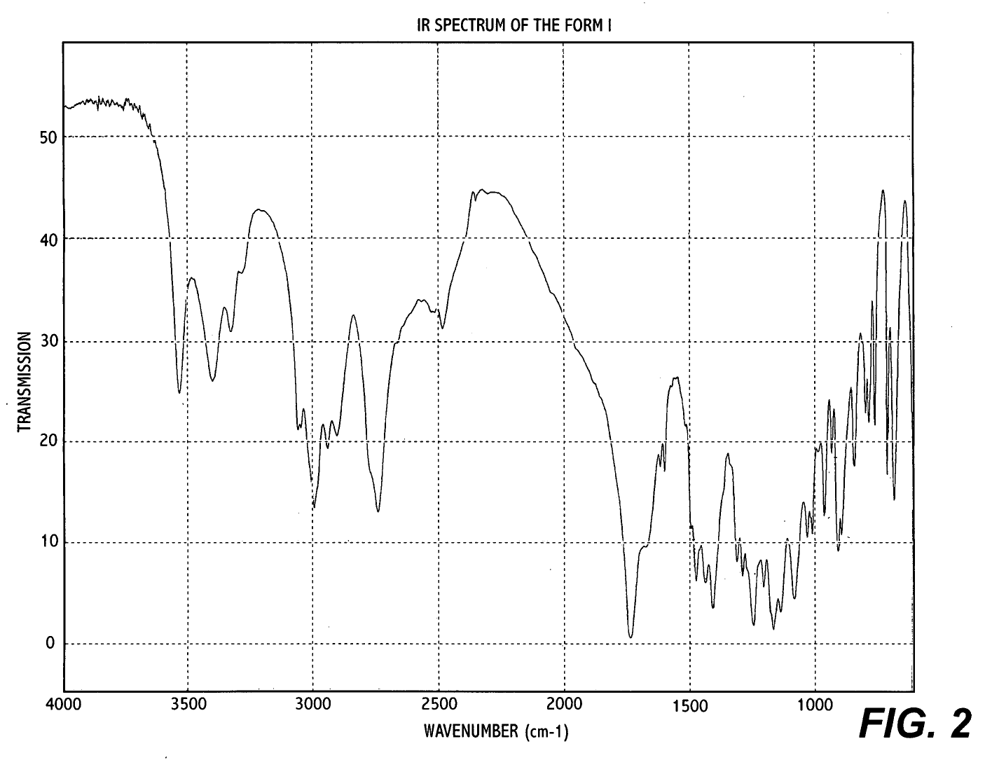

After repeating the conventional processes for making rivastigmine hydrogentartrate in solid state, it was determined that the conventional processes yield a crystalline rivastigmine hydrogentartrate, herein denoted as Form I. The crystalline rivastigmine hydrogentartrate Form I can generally be characterized by an XRPD pattern as shown in FIG. 1, and has characteristic diffraction peaks at the following angles: 5.2, 10.3, 11.9, 12.4, 14.8, 16.6, 17.3, 17.7, 18.8, 20.6, 21.2, 23.2, 24.9, 27.8, and 30.4° 2θ+/−0.1. Additionally or alternatively, the crystalline rivastigmine hydrogentartrate Form I can be characterized by an IR spectrum as shown in FIG. 2 and has characteristic reflections at IR absorbance peaks (wavenumbers) of 3530, 3397, 2736, 1728, 1399, 1280, and 755 cm⁻¹+/−5 cm⁻¹.

A second crystalline polymorph of rivastigmine hydrogentartrate and an amorphous rivastigmine hydrogentartrate, herein denoted as Form II and Form III, respectively, have surprisingly been discovered. The Form II is a new crystalline polymorph of rivastigmine hydrogentartrate having a different crystal structure from the Form I. The different crystal structures of Forms I and II can typically be identified by XRPD, although other methods such as IR, etc., may also be used.

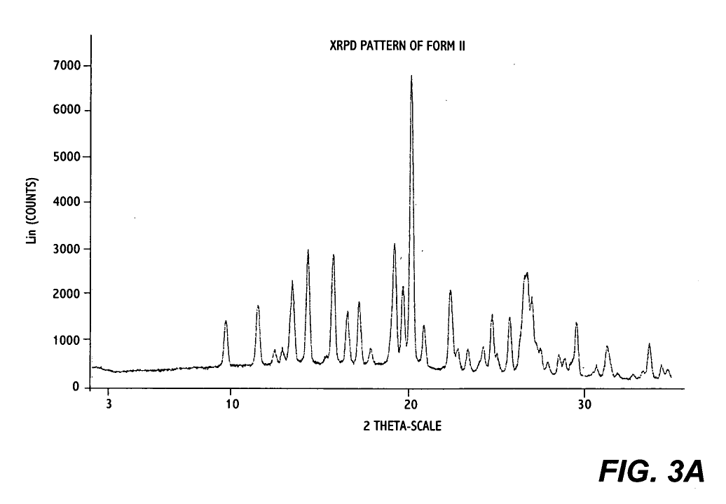

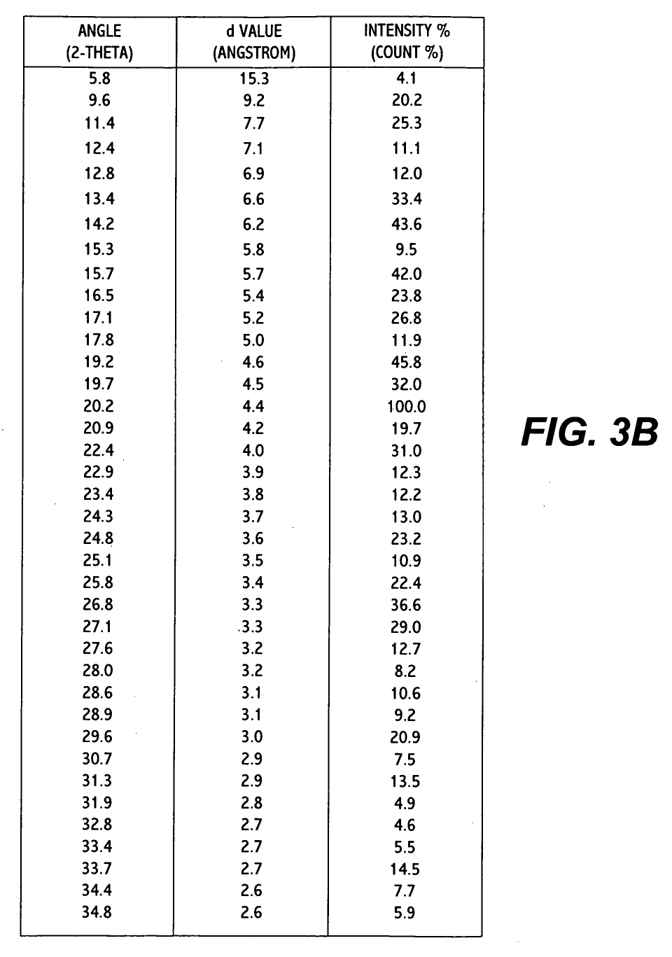

Form II crystal structure can be characterized by an XRPD pattern, which is distinct from that of the Form I. Generally, the Form II can be characterized by peaks in its XRPD pattern at angles of about 9.6, 11.4, 13.4, 19.2, 20.2, and 22.4°, and typically peaks of about 9.6, 11.4, 13.4, 15.7, 19.2, 20.2, 22.4, 24.8, and 29.6° 2θ+/−0.1°, and in most embodiments angles of about 9.6, 11.4, 13.4, 14.2, 15.7, 19.2, 20.2, 22.4, 24.8, 26.8, 29.6, 31.3, and 33.7° 2θ+/−0.1°. In particular, rivastigmine tartrate Form II generally exhibits large and/or significant peaks at around 9.6, 11.4, 13.4, 19.2, 20.2, and 22.4° 2θ+/−0.1° while the Form I exhibits no corresponding or significant peaks. Thus, the presence of peaks at around 9.6, 11.4, 13.4, 19.2, 20.2, 22.4, and 24.8, and optionally further at 15.7, 29.6° 2θ, can be used to characterize or identify the presence of the Form II crystal structure in a rivastigmine hydrogentartrate sample. Conversely, appreciable and/or large peaks in the XRPD pattern of the known Form I are present at angles of about 5.2, 14.8, 18.8, 20.6, and 21.2° 2θ+/−0.1°, but corresponding appreciable and/or large peaks are not present in the corresponding XRPD pattern of the Form II. Rivastigmine hydrogentartrate Form II exhibiting an XRPD pattern that substantially corresponds with FIG. 3 is a specific embodiment of the present invention. Generally, the measured angle values for rivastigmine hydrogentartrate Form I and Form II are within +/−0.1° of the above-recited values, more preferably the measured values are identical to the above values after truncating or rounding to the tenths place.

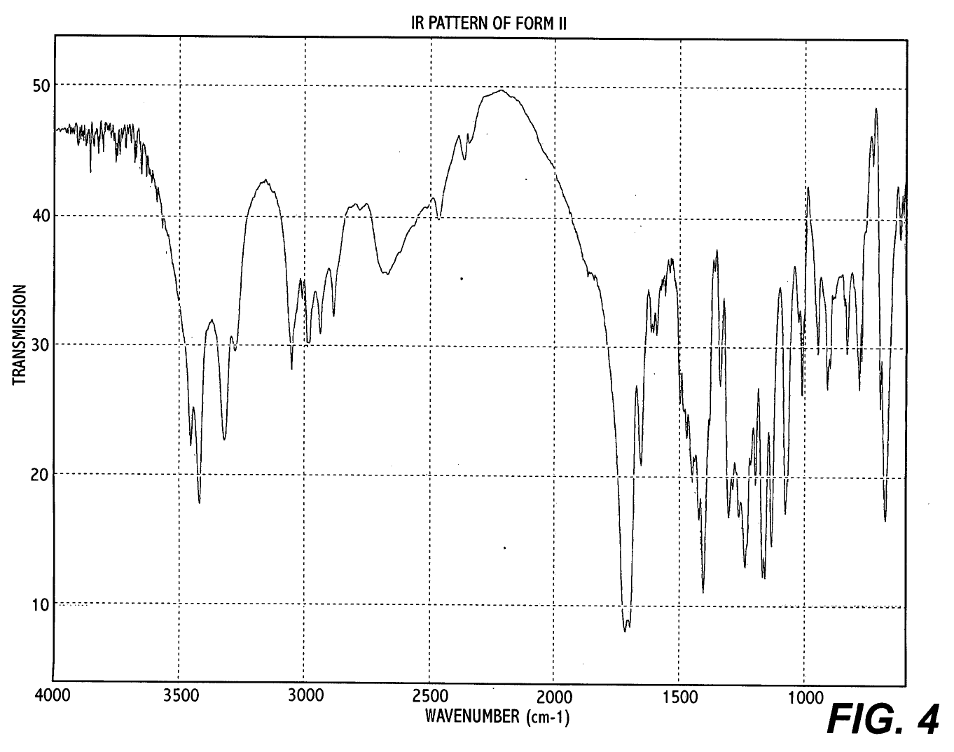

Somewhat surprisingly, the IR spectrum for the two forms is also different. As seen in comparing FIG. 2 with FIG. 4, rivastigmine hydrogentartrate Form II (FIG. 4) exhibits many differences in IR absorbance from the Form I (FIG. 2). Similarly, a rivastigmine hydrogentartrate Form II exhibiting an IR absorbance spectra that substantially corresponds to FIG. 4 is a specific embodiment of the present invention.

The phrases “substantially corresponds” and “substantially as shown in” each encompasses variations caused by different sample preparations, different equipment and/or settings used in measuring, normal experimental error/variation, and small amounts of impurities. Differences in a pattern or spectra that are not attributable to these factors indicate that the pattern or spectra in question does not “substantially correspond” to the reference pattern. Conversely, a spectrum may “substantially correspond” to another one even though it is not an identical, superimposable image. The identification of the Form II is not limited to x-ray powder diffraction or IR spectra. Any technique that can distinguish the Forms I and II, such as by different physical properties, can be used.

The present invention includes rivastigmine hydrogentartrate Form II as an isolated substance, especially in an essentially pure form and/or essentially morphologically pure form. “Essentially pure” refers to at least 70% pure, preferably at least 80% pure, more preferably at least 90% pure, and still more preferably at least 95%, including at least 98% pure, at least 99% pure, and at least 99.8% pure, with respect to the presence of rivastigmine hydrogentartrate in a sample. “Essentially morphologically pure” refers to at least 70% pure, preferably at least 80% pure, more preferably at least 90% pure, and still more preferably at least 95%, including at least 98% pure, at least 99% pure, and at least 99.8% pure, with respect to the presence of the Form II in a sample. Typically in order for a sample of rivastigmine hydrogentartrate Form II to have an XRPD pattern or IR spectrum that substantially corresponds to FIG. 3 or FIG. 4, respectively, the Form II sample must be in essentially pure form and essentially pure morphological form (typically at least 90% pure morphologically).

The present invention also includes mixtures of the Form II with other forms of rivastigmine hydrogentartrate, such as with the Form I and/or with amorphous rivastigmine hydrogentartrate (Form III, discussed below). The different forms of rivastigmine hydrogentartrate are present in the mixture in sufficient amounts to be detected, for example, using XRPD. That is, different forms of rivastigmine hydrogentartrate can be distinguished based on their respective different XRPD patterns. Thus, a composition that contains a small amount or a large amount of the Form II, regardless of the other materials/substances optionally present therewith, is also contemplated to be part of the present invention, but the essentially pure composition is preferred.

The rivastigmine hydrogentartrate molecule can be made by synthesis techniques well known in the art, including (but not limited to) the processes mentioned in the above-identified patents. As recited above, the conventional processes provide, after a crystallization and/or a precipitation, the known Form I.

The Form II of rivastigmine hydrogentartrate may be made by liquid-mediated solid-solid transformation of the Form I. In one embodiment, the Form II may be made by contacting, preferably under stirring, solid rivastigmine hydrogentartrate Form I with the liquid mediator for a prolonged time (optionally for a period longer than 3 hours) and at a suitable temperature (which preferably does not exceed 50° C. and is most preferably ambient temperature). Depending on the duration of the contacting step, a mixture of the Form I and Form II may be obtained, in which the relative amount of the Form II increases with an increase in the length of time of the contact and may reach a state at which the product is essentially pure Form II (i.e., substantially free from the Form I).

Suitable liquid mediators for making the Form II by the above process include (but are not limited to) ethyl acetate and isopropanol, alone or in combination. In an advantageous arrangement, a seeding crystal of the Form II may be used to facilitate the transformation. Alternatively, starting with a mixture of forms can have a similar effect as adding a seeding crystal in terms of speed, efficiency and/or completeness of conversion. Such a mixture of forms could be the result, for example, of the crystallization/precipitation of the rivastigmine hydrogentartrate. In this way, a sample can be converted to an essentially morphologically pure rivastigmine hydrogentartrate Form II, whether it initially contained only small of amounts of Form II (e.g. 20% or less) or large amounts of Form II (e.g. greater than 50%).

The solid rivastigmine hydrogentartrate product made according to the above process may be isolated from the liquid mediator by a conventional process, such as by filtration or centrifugation, and optionally washed by a suitable liquid and dried. The dried product may be further milled and/or sieved.

The amorphous rivastigmine hydrogentartrate Form III may be made by freeze-drying a solution including rivastigmine in aqueous solvent. In this respect, any kind or form of rivastigmine hydrogentartrate, including mixtures of forms (e.g., a mixture of Forms I and II), may be used as the starting material.

Similarly as disclosed above for the Form II, the present invention includes rivastigmine hydrogentartrate Form III as an isolated substance, especially in an essentially pure form. The present invention also includes mixtures of the Form III with other forms of rivastigmine hydrogentartrate, especially with the Form I and/or with the Form II.

Rivastigmine hydrogentartrate Form II and/or Form III can be formulated into various pharmaceutical compositions with one or more pharmaceutically-acceptable excipients. The pharmaceutical composition can be in a unit dosage form, such as a solid oral dosage form (i.e. tablet or capsule), a solution or suspension (especially for an aqueous sterile solution or suspension for parenteral administration), or bulk precursors thereof, such as a pre-blended mixture ready for further blending/addition of ingredients or a blend ready for tabletting or filling into capsules.

Usually the excipient is a pharmaceutically-acceptable carrier or diluent, such as one or more calcium phosphates, microcrystalline cellulose, hydroxypropyl methylcellulose, lactose, and starches, but is not limited thereto. In some embodiments, a polymer that is able to form a molecular dispersion with rivastigmine hydrogentartrate is used as an excipient. An example of such a polymer is polyvinylpyrrolidone hydroxypropylmethylcellulose phthalate. Such a dispersion can be formed by methods well known in the art, for example, dissolving the rivastigmine hydrogentartrate and the polymer in a suitable solvent and evaporating the solvent. Other excipients include fillers, binders, lubricants, disintegrants, preservatives, pH-adjustors, colorants, etc.

The pharmaceutical compositions are preferably formulated into tablets. The tablets may be monolithic tablets, i.e. tablets that upon ingestion do not disintegrate into a plurality of smaller units from which the active ingredient is finally released, or may be disintegrable tablets. The tablets may be produced by any standard tabletting technique, e.g. by wet granulation, dry granulation, or direct compression. Tabletting methods that do not employ a solvent (“dry processes”) are preferable. The tablet compositions may be further coated by a film coat. The film coat may protect the tablet against the environment (light, air, moisture) during storage and handling. Any conventional film coat may be used.

Alternatively, rivastigmine hydrogentartrate pharmaceutical compositions can be filled into capsules. Generally, the process comprises blending the active substance and excipients in one or more mixing or blending steps and then filling the blend into capsules.

The pharmaceutical compositions of the present invention contain rivastigmine hydrogentartrate Form II and/or Form III, as either the only rivastigmine hydrogentartrate form or as one of two or more forms. In a particular embodiment, the pharmaceutical composition is substantially free of the rivastigmine hydrogentartrate Form I, i.e. the pharmaceutical composition contains less than 2%, more preferably less than 1%, of Form I relative to the sum of all forms therein. In another embodiment, the pharmaceutical composition contains a mixture of forms (such as rivastigmine hydrogentartrate Form I and Form II), in which the relative amount of the form other than Form I (e.g. the Form II) is within the range of 1% to 100%, based on the total weight of all forms of rivastigmine hydrogentartrate. Typically at least 10%, and preferably at least 90% of the rivastigmine hydrogentartrate is Form II.

The pharmaceutical compositions of the present invention are normally formulated into unit dosage forms such as the above-described tablets or capsules. In a unit dosage form, the total amount of rivastigmine hydrogentartrate present, regardless of form, is effective for providing a therapeutic effect to a mammal. The unit dose may be a single tablet, one half of a tablet, or two or more tablets taken at essentially the same time or in the same administration. Unit dose in capsule form may comprise one or more capsules.

The rivastigmine hydrogentartrate containing at least a portion of rivastigmine hydrogentartrate Form II (e.g., at least 0.1%) can be used to treat a mammal in need thereof by administering a therapeutically effective amount of the rivastigmine hydrogentartrate.

The present invention is further described with reference to the following non-limiting examples.

### EXAMPLES

### Reference Example

### Form I

2 g S-(−)-Rivastigmine and 1.2 g L-(+)-tartaric acid were added to 20 ml acetone and the mixture was heated at 60° C. for 1 hour to get a clear mixture. The solution was allowed to slowly cool to ambient temperature and was stirred for 18 hours at 4° C. until complete precipitation of the tartrate salt. The solid was isolated by filtration, washed with acetone, and dried for 22 hours under vacuum at 40° C.

Isolated yield: 2.72 g, white solid

XRPD pattern: comparable to FIG. 1.

### Example 1

### Form II

0.5 g Rivastigmine hydrogentartarte Form I was suspended in 25 ml ethyl acetate. The suspension was stirred at ambient temperature and seeds from Form II were added. The suspension was stirred at ambient temperature for 24 hours. The white solid was isolated by filtration and air dried for 72 hours. A corresponding XRPD pattern shows a mixture of Forms I and II. The solid was suspended in 25 ml ethyl acetate. The suspension was stirred at ambient temperature for 72 hours. The white solid was isolated by filtration and dried at air for 17 hours.

XRPD pattern: comparable to FIG. 3.

IR spectrum: corresponds to FIG. 4.

### Example 2

### Amorphous Rivastigmine Hydrogentartrate

### Form III

0.5 g Rivastigmine hydrogentartrate Form I was dissolved in 10 ml water. The solution was freeze-dried, resulting in a white solid material. The sample was analyzed by XRPD directly after freeze-drying.

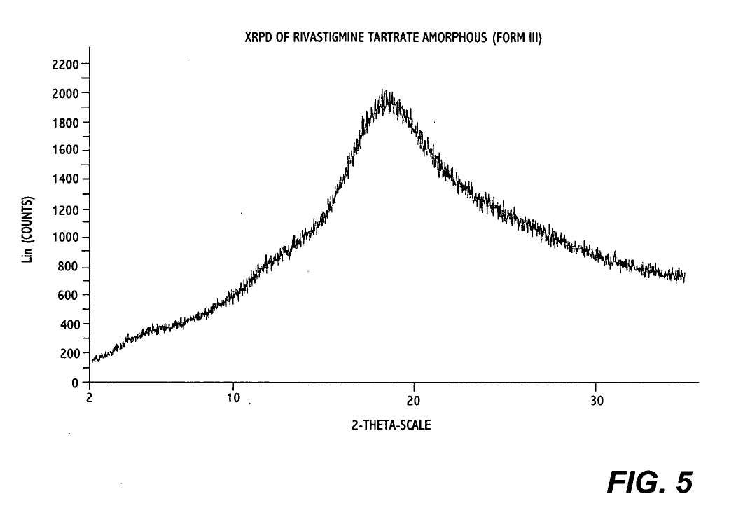

XRPD pattern: shows the material is amorphous (see FIG. 5).

Each of the patents mentioned above are incorporated herein by reference in their entirety. The invention having been described, it will be obvious that the same may be varied in many ways and all such modifications are contemplated as being within the scope of the invention as defined by the following claims.

1. A crystalline rivastigmine hydrogentartrate of Form II.

2. The rivastigmine hydrogentartrate according to claim 1, having an XRPD pattern with peaks at angles of about 9.6, 11.4, 13.4, 19.2, 20.2, and 22.4° 2θ+/−0.1°.

3. The rivastigmine hydrogentartrate according to claim 2, wherein the XRPD pattern includes peaks at angles of about 9.6, 11.4, 13.4, 15.7, 19.2, 20.2, 22.4, 24.8, and 29.6° 2θ+/−0.1°.

4. The rivastigmine hydrogentartrate according to claim 3, wherein the XRPD pattern includes peaks at angles of about 9.6, 11.4, 13.4, 14.2, 15.7, 19.2, 20.2, 22.4, 24.8, 26.8, 29.6, 31.3, and 33.7° 2θ+/−0.1°.

5. The rivastigmine hydrogentartrate according to claim 2, wherein the XRPD pattern does not include peaks at angles of about 5.2, 14.8, 18.8, 20.6, and 21.2° 2θ+/−0.1°.

6. The rivastigmine hydrogentartrate according to claim 1, wherein said rivastigmine hydrogentartrate exhibits an XRPD pattern substantially corresponding to FIG. 3A.

7. The rivastigmine hydrogentartrate according to claim 1, having IR absorbance peaks at 3455, 3422, 3321, 1717, 1699, 1655, 1406, 1338, and 948 cm^{-1+/-5} cm⁻¹.

8. The rivastigmine hydrogentartrate according to claim 1, exhibiting an IR absorbance spectrum substantially corresponding to FIG. 4.

9. The rivastigmine hydrogentartrate according to claim 1, wherein said rivastigmine hydrogentartrate is at least 90% pure Form II rivastigmine hydrogentartrate.

10. An amorphous rivastigmine hydrogentartrate of Form III.

11. A rivastigmine composition comprising a mixture of crystalline rivastigmine hydrogentartrate of Form II and at least one of crystalline rivastigmine hydrogentartrate of Form I and amorphous rivastigmine hydrogentartrate, wherein the mixture of forms can be detected by XRPD.

12. A pharmaceutical composition comprising an effective amount of the rivastigmine composition according to claim 11 and a pharmaceutically-acceptable excipient.

13. A pharmaceutical composition comprising the rivastigmine hydrogentartrate of Form II according to claim 1, and a pharmaceutically-acceptable excipient.

14. The pharmaceutical composition according to claim 13, wherein said composition is a unit dose and said rivastigmine hydrogentartrate of Form II is contained in an effective amount.

15. The pharmaceutical composition according to claim 14, wherein said composition is substantially free of Form I rivastigmine hydrogentartrate.

16. The pharmaceutical composition according to claim 15, wherein said pharmaceutically acceptable excipient is a carrier or a diluent.

17. The pharmaceutical composition according to claim 16, wherein said composition is a solid oral dosage form.

18. A process, which comprises contacting solid state rivastigmine hydrogentartrate with a liquid sufficiently to form rivastigmine hydrogentartrate Form II.

19. The process according to claim 18, wherein the contacting step is performed at a temperature lower than 50° C.

20. The process according to claim 19, wherein a duration of the contacting step is at least 3 hours.

21. A process which comprises forming amorphous rivastigmine hydrogentartrate by freeze drying a solution of rivastigmine hydrogentartrate in water.
